(19) United States
(12) Patent Application Publication
(10) Pub. No.: US 2005/0137461 A1
(43) Pub. Date: Jun. 23, 2005

(54) Telescoping blade assembly and instruments for adjusting an adjustable blade

(75) Inventors: Connie Marchek, Foxborough, MA (US); Thomas Higginbotham, Independence, MO (US); Douglas Raymond, Quincy, MA (US); Michael Mahoney, Middletown, RI (US); William Frasier, New Bedford, MA (US); Anthony Carlone, Bristol, RI (US); Paul Maguire, Hope Valley, RI (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DePuy Spine, Inc., Raynham, MA (US)

(21) Appl. No.: 10/815,182

(22) Filed: Mar. 31, 2004

Related U.S. Application Data
(60) Provisional application No. 60/530,565, filed on Dec. 18, 2003.

(51) Int. Cl. A61B 1/32

(52) U.S. Cl. 600/220

(57) ABSTRACT

A retractor blade assembly includes a fixed blade having a longitudinal axis and an adjustable blade operatively coupled to the fixed blade and adjustable relative to the fixed blade along the longitudinal axis of the fixed blade. The adjustable blade may include a flexible tab that is movable between a first position, in which the tab is generally co-planar to the adjustable blade, and a second position, in which the tab is oriented generally transverse to the adjustable blade.

### CROSS-REFERENCE TO RELATED APPLICATIONS

This application claims priority to U.S. Provisional Patent Application Ser. No. 60/530,565, filed Dec. 18, 2003, entitled Surgical Retractor Systems, Illuminated Cannula, and Method of Use, which is incorporated herein by reference

### BACKGROUND

In surgical procedures, it is important to minimize trauma to the patient and damage to tissue to facilitate patient recovery. One way to accomplish this is to minimize the size of the incision for the surgical procedure. A number of retractors are available that are designed to expand a small surgical incision and provide access to a surgical site. Such retractors often require the surgeon to select retractor blades of an appropriate length and width for the patient's anatomy during the surgical procedure. As a result, conventional retractors often include a large set of retractor blades of varying fixed lengths and widths for use with the retractor. Such fixed retractor blades may not conform to each patient's individual anatomy.

### SUMMARY

Disclosed herein are retractor blade assemblies that facilitate the retraction of soft tissue during a surgical procedure and may be adjusted to conform to varying patient anatomy. The disclosed retractor blade assemblies may be used with a wide range of surgical retractors and in any type of surgical procedure in which a retractor may be employed, including, for example, minimally invasive spine surgery. Also disclosed herein are surgical instruments that may be employed to easily adjust an adjustable blade of a retractor blade assembly, such as one of the retractor blade assemblies disclosed herein.

In one exemplary embodiment, a retractor blade assembly may comprise a fixed blade having a longitudinal axis and an adjustable blade operatively coupled to the fixed blade and adjustable relative to the fixed blade along the longitudinal axis of the fixed blade. In the exemplary embodiment, the adjustable blade may include a flexible tab that is movable between a first position, in which the tab is generally co-planar to the adjustable blade, and a second position, in which the tab is oriented generally transverse to the adjustable blade.

In another exemplary embodiment, a retractor blade assembly may comprise a fixed blade having a longitudinal axis, an adjustable blade operatively coupled to the fixed blade and adjustable relative to the fixed blade along the longitudinal axis of the fixed blade, and a flexible sheath coupled at one portion to the fixed blade and at a second portion to the adjustable blade.

In a further exemplary embodiment, a retractor blade assembly may comprise a fixed blade having a longitudinal axis and a plurality of adjustable blades operatively coupled to the fixed blade and adjustable relative to the fixed blade along the longitudinal axis of the fixed blade between a proximal position and a distal position. In the exemplary embodiment, the plurality of adjustable blades may include one or more laterally adjustable blades that are adjustable relative to one another in a direction transverse to the longitudinal axis of the fixed blade when the plurality of adjustable blades are adjusted to a distal position.

In one exemplary embodiment, an instrument for adjusting an adjustable blade of a retractor blade assembly may comprise a handle and an instrument body coupled to the handle. In the exemplary embodiment, the instrument body may include a distal end having a distal facing notch formed therein. The notch may be defined by a first surface and a second surface, which may be angled proximally toward the first surface. In the exemplary embodiment, the notch may be sized to receive a portion of the adjustable blade between the first surface and the second surface.

### BRIEF DESCRIPTION OF THE FIGURES

These and other features and advantages of the retractor blade assemblies, instruments, and methods disclosed herein will be more fully understood by reference to the following detailed description in conjunction with the attached drawings in which like reference numerals refer to like elements through the different views. The drawings illustrate principles of the retractor blade assemblies and instruments disclosed herein and, although not to scale, show relative dimensions.

FIGS. 1 and 2 are perspective views of an exemplary embodiment of a retractor blade assembly;

FIG. 3 is a perspective view of the fixed blade of the retractor blade assembly of FIGS. 1 and 2;

FIG. 4A is a perspective view of the inner surface of the adjustable blade of the retractor blade assembly of FIGS. 1 and 2, illustrating the flexible tab in a first position;

FIG. 4B is a side elevational view of the proximal end of the adjustable blade of FIG. 4A, illustrating the flexible tab in a second position;

FIG. 5 is a perspective view of the outer surface of the adjustable blade of the retractor blade assembly of FIGS. 1 and 2;

FIG. 6 is a perspective view of the outer surface of the retractor blade assembly of FIGS. 1 and 2;

FIG. 7 is a perspective view of the proximal end of the inner surface of the adjustable blade of FIG. 4;

FIG. 8A is a perspective view of another exemplary embodiment of a retractor blade assembly;

FIG. 8B is a side elevational view of the proximal end of the fixed blade of the retractor blade assembly of FIG. 8A;

FIG. 8C is a perspective view of the proximal end of the fixed blade of the retractor blade assembly of FIG. 8A, illustrating an instrument for adjusting the adjustable arm of the fixed blade;

FIG. 8D is a side elevational view of the distal end of the instrument of FIG. 8C;

FIG. 9 is a perspective view of an adjustable blade of the retractor blade assembly of FIG. 8A;

FIG. 10 is a perspective view of another exemplary embodiment of a retractor blade assembly;

FIG. 11 is a perspective view of the inner surface of the retractor blade assembly of FIG. 10;

FIG. 12 is a side elevational view in cross section of the retractor blade assembly of FIG. 10;

FIG. 13 is a top view in cross section of the retractor blade assembly of FIG. 10;

FIG. 14 is a perspective view of another exemplary embodiment of a retractor blade assembly, illustrating the adjustable blade in a proximal position;

FIG. 15 is a perspective view of the retractor blade assembly of FIG. 14, illustrating the adjustable blade in a distal position;

FIG. 16 is a perspective view of the inner surface of the retractor blade assembly of FIG. 14;

FIG. 17 is a perspective view of the adjustable blade of the retractor blade assembly of FIG. 14;

FIG. 18 is a perspective view of the inner surface of the adjustable blade of FIG. 17;

FIG. 19 is a perspective view of an exemplary embodiment of an instrument for adjusting the adjustable blade of a retractor blade assembly;

FIG. 20 is a perspective view of the instrument of FIG. 19, illustrating the distal end of the instrument engaging the adjustable blade of the retractor blade assembly of FIG. 1;

FIG. 21 is a perspective view of the distal end of the instrument of FIG. 19, illustrating the distal end engaging the adjustable blade of the retractor blade assembly of FIG. 1;

FIG. 22A is a perspective view of the distal end of the instrument of FIG. 19;

FIG. 22B is a side elevational view of the distal end of the instrument of FIG. 19;

FIG. 23 is a side elevational view of another exemplary embodiment of a retractor blade assembly including a flexible sheath, illustrating the adjustable blade in a proximal position;

FIG. 24 is a side elevational view of the retractor blade assembly of FIG. 23, illustrating the adjustable blade in a distal position;

FIG. 25 is a side elevational view of the inner surface of another exemplary embodiment of a retractor blade assembly, illustrating the adjustable blade in a proximal, laterally compact position;

FIG. 26 is a side elevational view of the inner surface of the retractor blade assembly of FIG. 25, illustrating the adjustable blade in a distal, laterally expanded position;

FIG. 27 is a bottom view of the retractor blade assembly of FIG. 25, illustrating the adjustable blade in a proximal, laterally compact position;

FIG. 28 is a side elevational view of the inner surface of the adjustable blade of the retractor assembly of FIG. 25, illustrating the adjustable blade in a laterally expanded configuration;

FIG. 29 is a side elevational view of the adjustable blade of the retractor assembly of FIG. 25, illustrating the adjustable blade in a laterally compact configuration;

FIG. 30 is a side elevational view of the distal end of an alternative exemplary embodiment of the adjustable blade of the retractor blade assembly of FIG. 25;

FIG. 31 is a side elevational view of the inner surface of the adjustable blade of another exemplary embodiment of a retractor blade assembly, illustrating the adjustable blade in a laterally compact configuration;

FIG. 32 is a side elevational view of the inner surface of the adjustable blade of FIG. 31 illustrating the adjustable blade in a laterally expanded configuration; and

FIG. 33 is a side elevational view of the inner surface of the adjustable blade of another exemplary embodiment of a retractor blade assembly.

### DETAILED DESCRIPTION

Certain exemplary embodiments will now be described to provide an overall understanding of the principles of the structure, function, manufacture, and use of the retractor blade assemblies and instruments disclosed herein. One or more examples of these embodiments are illustrated in the accompanying drawings. Those of ordinary skill in the art will understand that the retractor blade assemblies and instruments specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The term “distal” as used herein with respect to any component or structure will generally refer to a position or orientation that is proximate, relatively, to a surgical site within the body. Conversely, the term “proximal” as used herein with respect to any component or structure will generally refer to a position or orientation that is distant, relatively, to a surgical site within the body.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

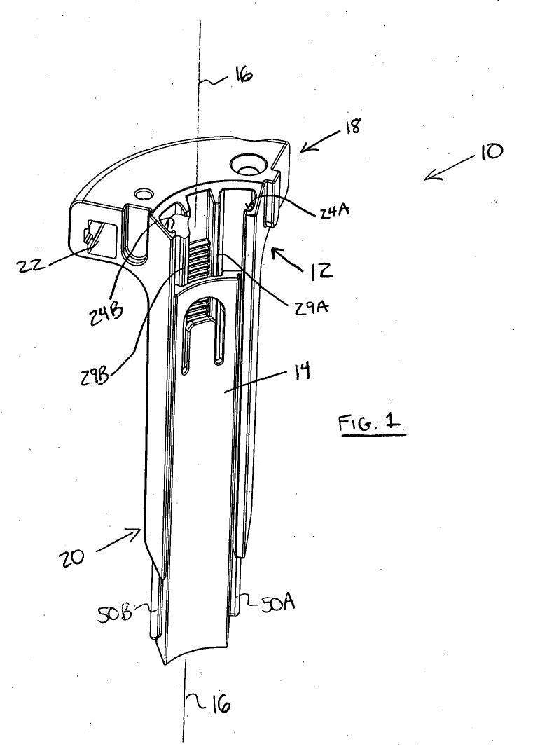

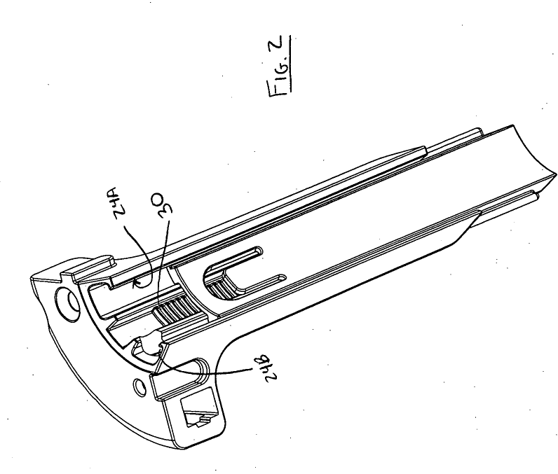

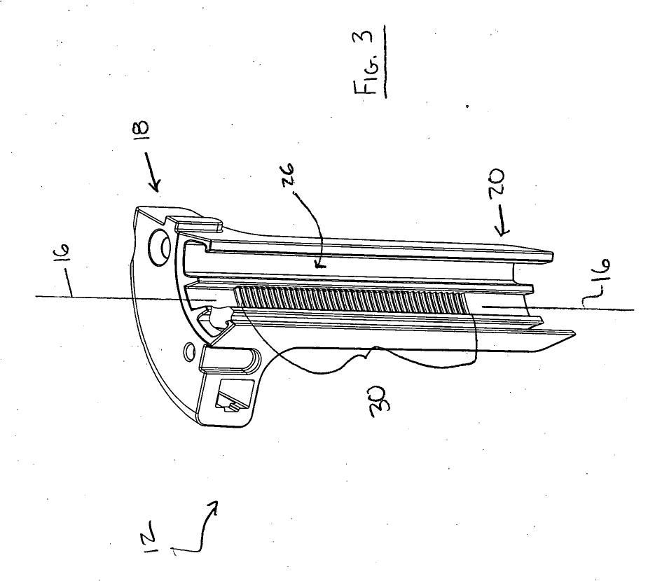

FIGS. 1-7 illustrate an exemplary embodiment of a retractor blade assembly 10 including a fixed blade 12 and an adjustable blade 14 that is operatively coupled to the fixed blade 12 and is adjustable relative to the fixed blade 12 along the longitudinal axis 16 of the fixed blade 12 between a proximal position and a distal position. The exemplary retractor blade assembly 10 may be used alone or in combination with other retractor blades to form a surgical retractor that is suitable for retracting tissue during a surgical procedure. For example, the exemplary surgical retractor blade assembly 10 may be employed in one or more of the retractors disclosed in commonly-owned U.S. Provisional Patent Application No. 60/530,565, entitled Surgical Retractor Systems, Illuminated Cannula, and Method of Use, which is incorporated herein by reference. The retractor blade assembly 10 may used to retract any type of tissue in any type of surgical procedure, including, for example, minimally invasive spine procedures, and to create a working channel from a skin incision to a surgical site in vivo.

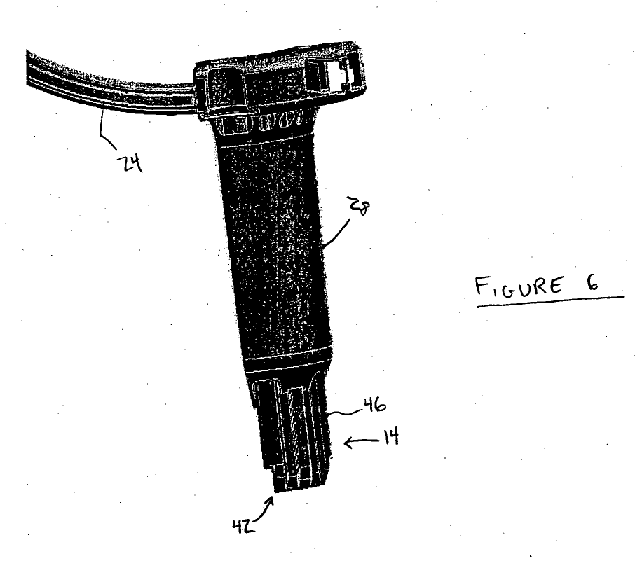

The fixed blade 12 of the exemplary retractor blade assembly 10 includes a proximal end 18 spaced apart along the longitudinal axis 16 from a distal end 20. The proximal end 18 may be configured to connect with a surgical retractor or with other retractor blade assemblies of similar or dissimilar constructions. In the exemplary embodiment, for example, the proximal end 18 includes an opening 22 for receiving an arm 24 (FIG. 6) of a retractor, such as a retractor disclosed in above-referenced provisional patent application. One skilled in the art will appreciate that the proximal end 18 may be alternatively configured, depending on the type of retractor, to facilitate connection with other types of retractors. The fixed blade 12 includes a pair of spaced apart channels 24A and 24B that extend longitudinally from the proximal end 18 to the distal end 20 of fixed blade 12. Each channel 24A, 24B is sized and shaped to receive a rail 50 provided on the adjustable blade 14, as discussed in more detail below. The channels 24A, 24B cooperate with the rails 50A, 50B to guide the adjustable blade 14 during adjustment relative to the fixed blade 12. The channels 24A, 24B are oriented parallel to one another and parallel to the longitudinal axis 16 of the fixed blade 12, although other orientations are possible depending on the desired path for the adjustable blade 14. In addition, the length of the channels 24A,B can be varied depending on, for example, the length of the adjustable blade 14 and the amount of longitudinal adjustment desired. The channels 24A, 24B need not extend along the entire length of the fixed blade 12, as in the illustrated embodiment.

The fixed blade 12 may optionally include one or more stops for fixing the adjustable blade 14 at a position along the longitudinal axis of the fixed blade 12. In the exemplary embodiment, for example, the fixed blade 12 includes a plurality of stops, in the form of teeth 30, aligned longitudinally along the fixed blade 12. The teeth 30 are configured to be engaged by a projection 72 provided on the adjustable blade 14, as discussed in more detail below. The number and position of the stops provided on the fixed blade 12 may be varied depending on the particular application. Moreover, the configuration of the stops may be varied. For example, each stop may be a hole, detent, rib, shoulder, or any other structure for fixing the adjustable blade 14, in a temporary or permanent manner, relative to the fixed blade 12. In other exemplary embodiments, one or more stops may be provided on the adjustable blade 14 in addition to or in place of one or more stops provided on the fixed blade 12.

Continuing to refer to FIGS. 1-7, the adjustable blade 14 of the exemplary embodiment includes a proximal end 40 spaced apart from a distal end 42. The adjustable blade 14 may include an inner surface 44 and an outer surface 46. The inner surface 44, in cooperation with the inner surface 26 of the fixed blade 12, can define a working channel for the retractor assembly 10. The outer surface 46 of the adjustable blade 14, in cooperation with the outer surface 28 of the fixed blade 12, can face and contact tissue to be retracted to inhibit the retracted tissue from entering or otherwise interfering with the working channel defined by the inner surfaces 26, 44 of the retractor blade assembly 10. The outer surface 46 of the adjustable blade 14 and the outer surface 28 of the fixed blade 12 may each include surface texturing, e.g., one or more grooves or surface roughening, or other surface treatments or coatings to improve gripping of retracted tissue. The adjustable blade 14 may include one or more guide rails 50 that cooperate with one or more channels 24 provided on the fixed blade 12. In the illustrated embodiment, for example, the adjustable blade 14 includes a pair of spaced apart guide rails 50A, 50B. Each of the guide rails 50A, 50B are sized to seat within a respective one of the channels 24A, 24B. As discussed above, the guide channels 50A, 50B cooperate with the channels 24A, 24B, to guide the adjustable blade 14 during adjustment relative to the fixed blade 12. Like the channels 24, the length, orientation, and number of rails 50 may be varied. The rails 50 need not extend along the entire length of the adjustable blade 14, as in the exemplary embodiment.

One skilled in the art will appreciate that the location of the channels 24 and guide rails 50 may reversed, e.g., the adjustable blade 14 may be provided with one or more channels 24 and the fixed blade 12 may be provided with one or more guide rails 50.

The outer surface 46 of the adjustable blade 14 may include one or more grooves 52 that engage the fixed blade 12 to facilitate alignment of the adjustable blade 14 relative to the fixed blade 12 and facilitate tissue engagement. In the illustrated embodiment, for example, the outer surface 46 includes a pair of longitudinally aligned, parallel grooves 52A, 52B that are sized and shaped to engage a pair of longitudinally aligned parallel ribs 29A, 29B provided on the fixed blade 12.

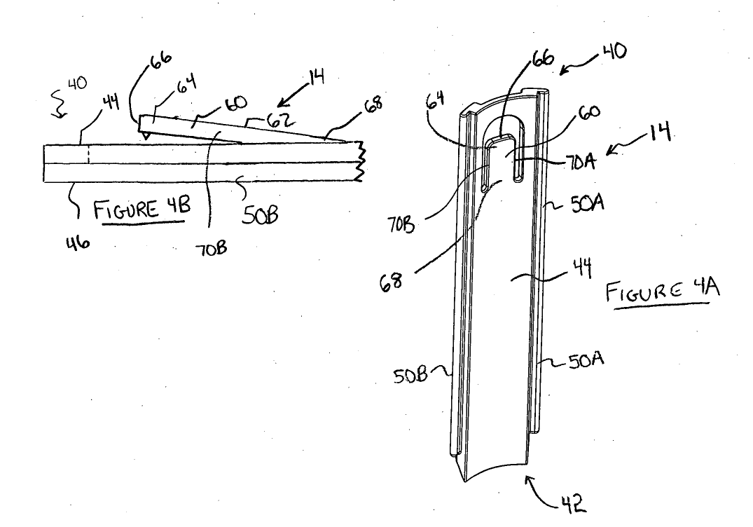

The adjustable blade 14 may include a flexible tab 60 that facilitates adjustment of the adjustable blade 14 relative to the fixed blade 12 and cooperates with one or more of stops provided on the fixed blade 12 to fix the adjustable blade 14 relative to the fixed blade 12. The flexible tab 60, in the exemplary embodiment, is movable between a first position, in which the tab 60 is generally co-planar to the adjustable blade 14 as illustrated in FIG. 4A, and a second position, in which the tab 60 is oriented generally transverse to the adjustable blade 14, as illustrated in FIG. 4B. In the first co-planar position, the inner surface 62 of the tab 60 is preferably aligned with the inner surface 44 of the adjustable blade 14 such that the tab 60 minimizes interference with the working channel created by the retractor blade assembly 10. The flexible tab 60, when in the first position, may be configured to engage to the fixed blade 12 to fix the adjustable blade 14 relative to the fixed blade 12, as discussed below. In certain embodiments, including the illustrated embodiment, the tab 60 may be biased to the first position. In the second position, as illustrated in FIG. 4B, the inner surface 62 of the tab 60 is oriented at an angle to the inner surface 44 of the adjustable blade 14. The angle of inner surface 62 in the second position may be varied depending on the configuration, e.g., the shape and material, of the flexible tab 60. In certain exemplary embodiments, the angle is greater than 3°. The flexible tab 60, when in the second position, may be configured to disengage the fixed blade 12 to facilitate adjustment of the adjustable blade 14 relative to the fixed blade 12. In such embodiments, the angle of the inner surface 62 may be selected to permit disengagement.

The flexible tab 60 may have a variety of shapes and sizes. In the illustrated embodiment, for example, the tab 60 is generally rectilinear in shape. The exemplary tab 60 includes a proximal end 64 having a proximally facing surface 66, a distal end 68, and two parallel, longitudinally aligned lateral surfaces 70A, 70B. The distal end 68, in the illustrated embodiment, is coupled to and formed from the adjustable blade 14. In this configuration, the tab 60 may pivot about the distal end 68 between the first position and the second position. The distal end 68 of the tab 60 need not be formed from the adjustable blade 14. In other exemplary embodiments, for example, the distal end 68, or other portions of the tab 60, may be separately constructed from the adjustable blade 14 and then coupled to the adjustable blade 14 by conventional fastening mechanisms. The tab 60 may be constructed from a flexible and resilient material such that the tab 60 may be deflected to the second position and, upon release, may return to the first position.

The tab 60 may include a projection 72 that is configured to engage the fixed blade 12, for example, one or more stops provided on the fixed blade 12, to fix the adjustable blade 14 relative to the fixed blade 12. The projection 72 may be sized and shaped to engage a stop provided on the fixed blade 12. In the exemplary embodiment, for example, the projection has a triangular shaped cross-section that is sized to engage one or more of the teeth 30 provided on the fixed blade 12. In other embodiments, the projection 72 may be spherical or hemispherical in shape to engage a hole or detent formed in the fixed blade 12. In the exemplary embodiment, the projection 72 engages one of the teeth 30 when the tab 60 is in the first position. When the tab 60 is moved away from the fixed blade 12 to a second position, the projection 72 may disengage the tooth, allowing the adjustable blade 14 to be adjusted relative to the fixed blade 12.

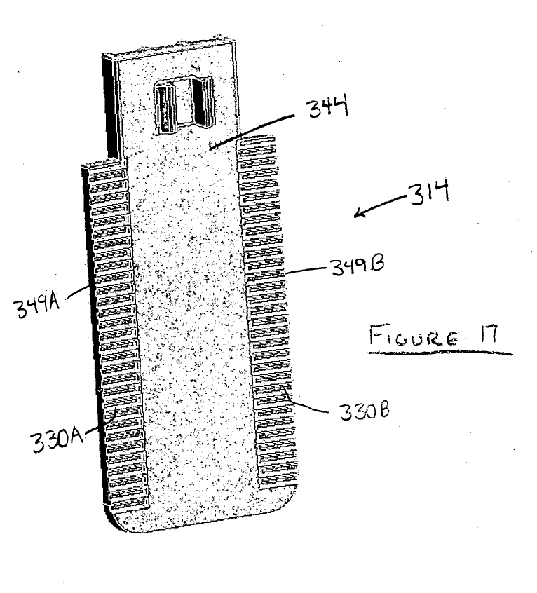

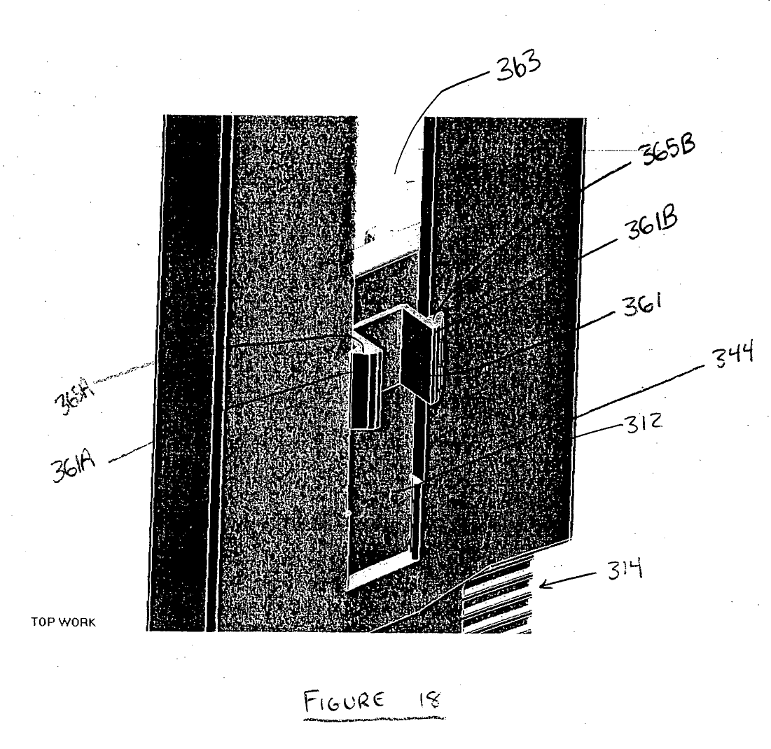

In certain exemplary embodiments, including the illustrated embodiment, a portion of the tab 60 may include an instrument engagement surface that is configured, e.g., sized, shaped, and/or oriented, to facilitate adjustment of the adjustable blade 14 by an instrument. In the illustrated embodiment, for example, the proximally facing surface 66 of the tab 60 is sized, shaped, and oriented to be engage by an instrument, such as the instrument illustrated in FIGS. 19-22, to facilitate adjustment of the adjustable blade 14. In particular, the proximally facing surface 66 of the tab 60 is oriented generally perpendicular to the longitudinal axis of the fixed blade 12.

Continuing to refer to FIGS. 1-7, the adjustable blade 14 may include an opening adjacent an instrument engagement surface to facilitate positioning of an instrument against the instrument engagement surface. In the illustrated exemplary embodiment, for example, an opening 74 through the adjustable blade 14, i.e., spanning between the inner surface 44 and the outer surface 46, is positioned proximal the proximally facing surface 66 of the flexible tab 60. The opening 74 may have a size and shape to receive a portion of instrument, such as the distal end of the instrument illustrated in FIGS. 19-22. For example, in the illustrated embodiment, the opening 74 has a length Lₒ and width Wₒ that is greater than or equal to a Lᵢ and Wᵢ of a portion of the instrument 500 illustrated in FIGS. 19-22, as discussed in further detail below. The opening 74 allows positioning of a portion of the distal tip of the instrument 500 between the flexible tab 60 and the fixed blade 12.

The fixed blade 12 and the adjustable blade 14 may be constructed from any material suitable for use in vivo, including, for example, metal, such as stainless steel or titanium, polymers, ceramics, or combinations thereof. In certain exemplary embodiments, the fixed blade 12 may be constructed from stainless steel and the adjustable blade may be constructed from a polymer.

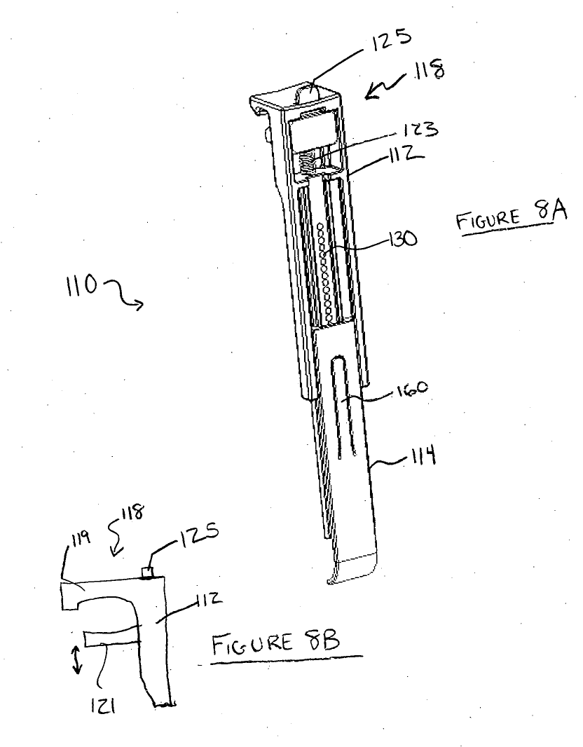

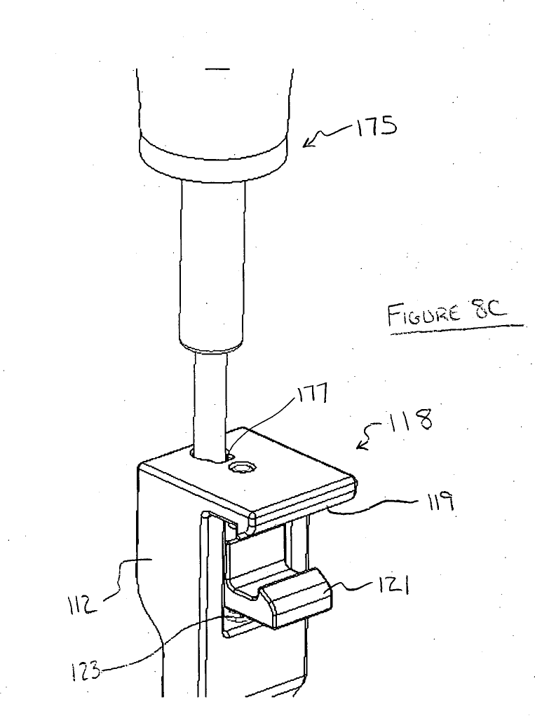

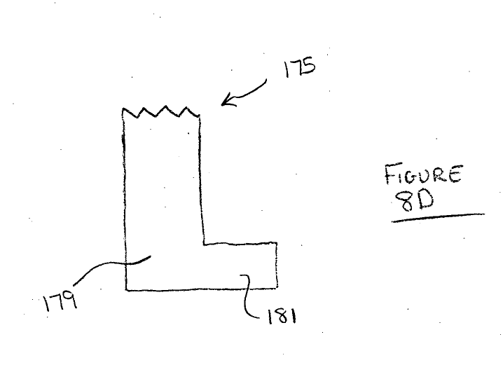

FIGS. 8-9 illustrate another exemplary embodiment of a retractor blade assembly 110 having a fixed blade 112 and an adjustable blade 114 that is adjustable relative to the fixed blade 112 along the longitudinal axis of the fixed blade 112. In the illustrated embodiment, the proximal end 118 of the fixed blade 112 is configured to engage a portion of a retractor, such as an arm 24 of a retractor. In particular, the proximal end 118 includes a fixed arm 119, which is oriented generally transverse to the longitudinal axis of the fixed blade 112, and an adjustable arm 121, which is oriented generally parallel to the longitudinal axis of the fixed blade 112 and is adjustable relative to the fixed arm 119 to selectively engage a portion of a retractor, such as the arm 24 of the retractor, therebetween. In particular, the adjustable arm 121 is adjustable between a closed position, illustrated in FIG. 8A, in which the distance between the fixed arm 119 and the adjustable arm 121 is less than or equal to the portion of the retractor to be engaged, and an open position in which the distance between the fixed arm 119 and the adjustable arm 121 is greater than the portion of the retractor to be engaged. In the exemplary embodiment, the adjustable arm 121 is biased to a closed position by a spring 123 or other biasing mechanism. A button 125, lever, handle or other adjustment mechanism may be provided to facilitate adjustment of the adjustable arm 121, as illustrated in FIGS. 8A and 8B. In other exemplary embodiments, an instrument 175 for adjusting the adjustable arm 121 relative to the fixed arm 119 may be provided, as illustrated in FIGS. 8C-D. For example, the proximal end 118 of the fixed blade 112 may include an opening 177 for receiving the distal end 179 of the instrument 175. The opening 177 may communicate with the adjustable arm 121 to allow a portion of the distal end 179 to contact the adjustable arm 121 to advance the adjustable arm 121 away from the fixed arm 119. The distal end 179 may have an L-shaped cross section including a base section 181 that extends generally transverse to the longitudinal axis of the instrument 175. The opening 177 may be generally key-shaped to allow the distal end 179 to pass through in one orientation and upon rotation of the instrument 175, the base section 181 may engage a ledge or other structure to fix the adjustable blade 121 in a desired position.

In the illustrated embodiment, the fixed blade 112 includes a plurality of stops in the form of detents 130. The detents 130 are longitudinally aligned and may be sized to receive a projection provided on a flexible tab 160.

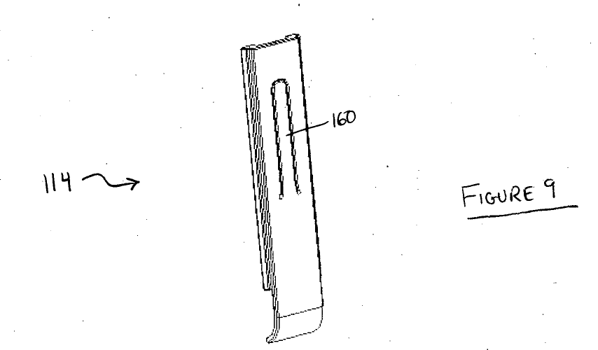

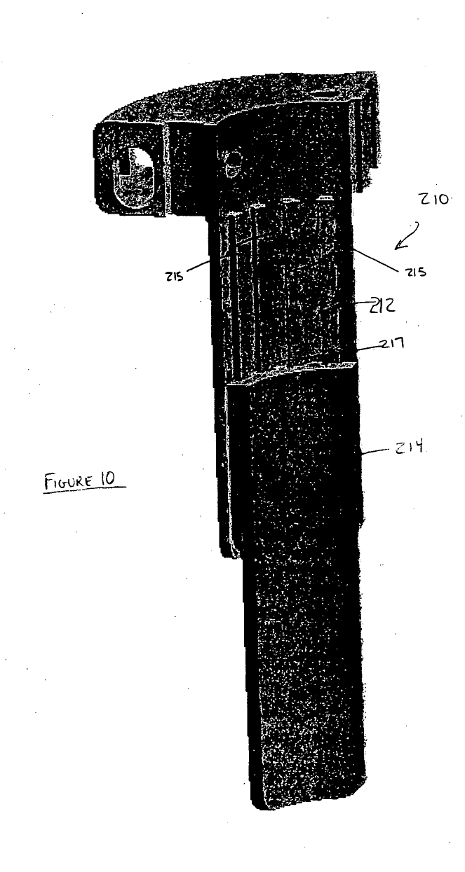

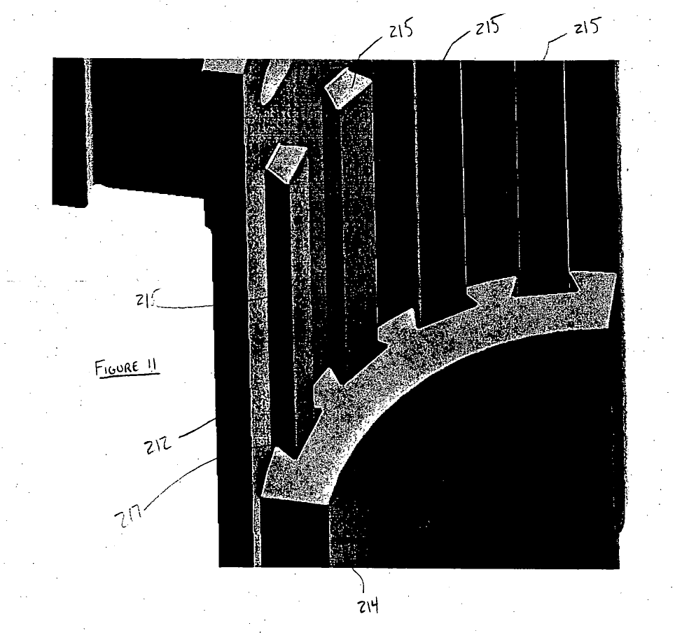

FIGS. 10-13 illustrate another exemplary embodiment of a retractor blade assembly 210 having a fixed blade 212 and an adjustable blade 214 that is longitudinal adjustable relative to the fixed blade 212. In the illustrated embodiment, the fixed blade 212 includes a plurality of longitudinally aligned parallel guide rails 215 that cooperate with a plurality of longitudinally aligned parallel slots 217 provided on the adjustable blade 214 to guide the adjustable blade 214 during adjustment relative to the fixed blade 212. The rails 215 may be complementary in shape to the grooves 217, as in the illustrated embodiment. In the illustrated embodiment, each rail 215 and each corresponding groove has a generally trapezoidal cross section. In this configuration, each rail 215 and each groove 217 interlock in the manner of a dovetail joint to limit the motion of the adjustable blade 214 to one direction, parallel to the longitudinal axis of the fixed blade 212. Although each rail 215 (and each groove 217) is similar in size and shape in the illustrated exemplary embodiment, one skilled in the art will appreciate that the fixed blade 212 may have rails 215 of varying size and shape and the adjustable blade 214 may have grooves 217 of varying size and shape. For example, in certain exemplary embodiments, the fixed blade 212 may have one rail with a trapezoidal cross section and one or more other rails with a different, e.g., non-trapezoidal, cross section.

In the illustrated embodiment, the fixed blade 212 may include a stop in the form of a ridge 230 provided at the distal end of the fixed blade 212. The ridge 230, in the illustrated embodiment, is oriented generally perpendicular to the longitudinal axis of the fixed blade 212, although other orientations are possible. The ridge 230 may span the width of the fixed blade 212, as in the illustrated embodiment, or may extend along a portion or portions of the width of the fixed blade 212. The adjustable blade 214 may include a shoulder 272 that engages the ridge 230 to limit the extent of longitudinal advancement of the adjustable bladed 214.

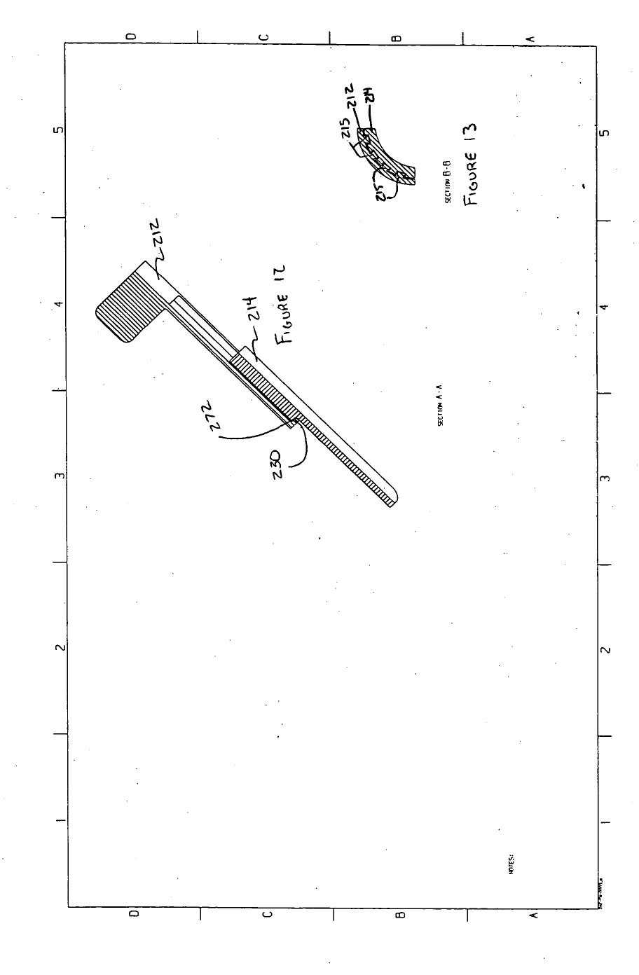

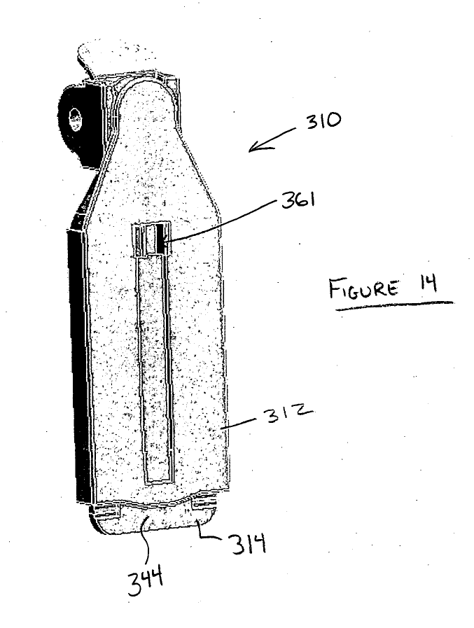

FIGS. 14-18 illustrate another exemplary embodiment of a retractor blade assembly 310 including a fixed blade 312 and an adjustable blade 314 that is adjustable along the longitudinal axis of the fixed blade 312. FIG. 14 illustrates the adjustable blade 314 in a proximal, retracted position in which a substantial portion of the adjustable blade 314 is positioned within the fixed blade 312. FIGS. 15 and 16 illustrate the adjustable blade 314 in a distal, expanded position in which a portion of the adjustable blade 314 is withdrawn from the fixed blade 312 thereby increasing the overall length of the retractor blade assembly 310.

Referring in particular to FIG. 16, the fixed blade 312 includes a pair of spaced apart channels 324A and 324B that extend longitudinally from the proximal end 318 to the distal end 320 of fixed blade 312. Each channel 324A, 324B is sized and shaped to receive a portion of the adjustable blade 314, such as a lateral edge 349A, 349B of the adjustable blade 314 as in the illustrated embodiment. In the illustrated embodiment, the channels 324A, 324B engage the lateral edges 349A, 349B to limit the motion of the adjustable blade 314 to longitudinal motion during adjustment relative to the fixed blade 312. The channels 324A, 324B are oriented parallel to one another and parallel to the longitudinal axis of the fixed blade 312, although other orientations are possible depending on the desired path for the adjustable blade 314. In addition, the length of the channels 324A,B can be varied depending on, for example, the length of the adjustable blade 314 and the amount of longitudinal adjustment desired. The channels 324A, 324B need not extend along the entire length of the fixed blade 312, as in the exemplary embodiment, but instead may extend along a portion or discrete portions of the fixed blade 312.

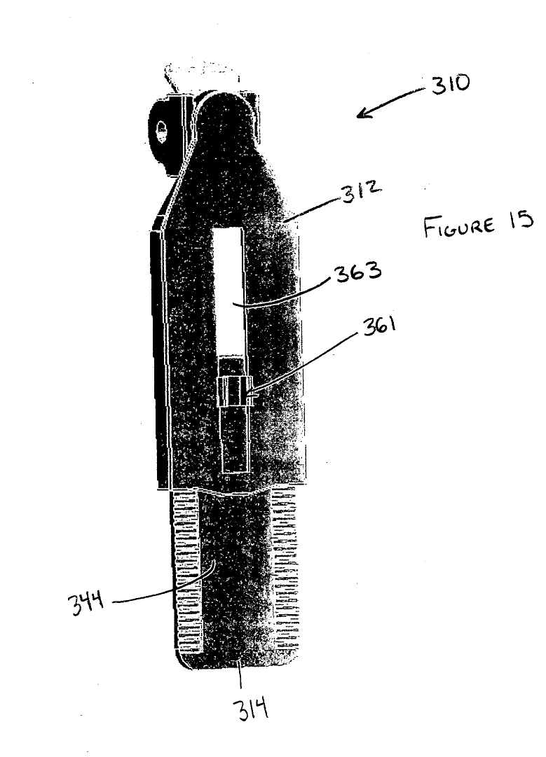

Referring in particular to FIG. 17, the inner surface 344 of the adjustable blade 314 may includes a plurality of stops, in the form of teeth 330, aligned longitudinally along the adjustable blade 314. In the illustrated embodiment, for example, two parallel columns of teeth 330A, B are provided on the lateral edges 349A, 349B, respectively, of the adjustable blade 314. The teeth 330 are configured to be engaged by a projection provided on the fixed blade 312. For example, in the illustrated embodiment, each channel 324A,B of the fixed blade 312 may be provided with a projection (not shown), provided, for example, at the distal end of each channel 324A, B. Each projection may engage one of the teeth 330 to fix the adjustable blade 314 in a position relative to the fixed blade 312.

In certain exemplary embodiments, the outer surface 346 of the adjustable blade 314 and the outer surface 328 of the fixed blade 312 may each include surface texturing, e.g., one or more grooves or surface roughening, or other surface treatments or coatings to improve gripping of retracted tissue. In the illustrated embodiment, for example, the outer surface 346 of the adjustable blade 314 may include one or more grooves 352 to facilitate tissue engagement. The grooves 352 may be aligned longitudinally along the length of the adjustable blade 314, as in the exemplary embodiment, or in other orientations or length(s) suitable to facilitate tissue engagement.

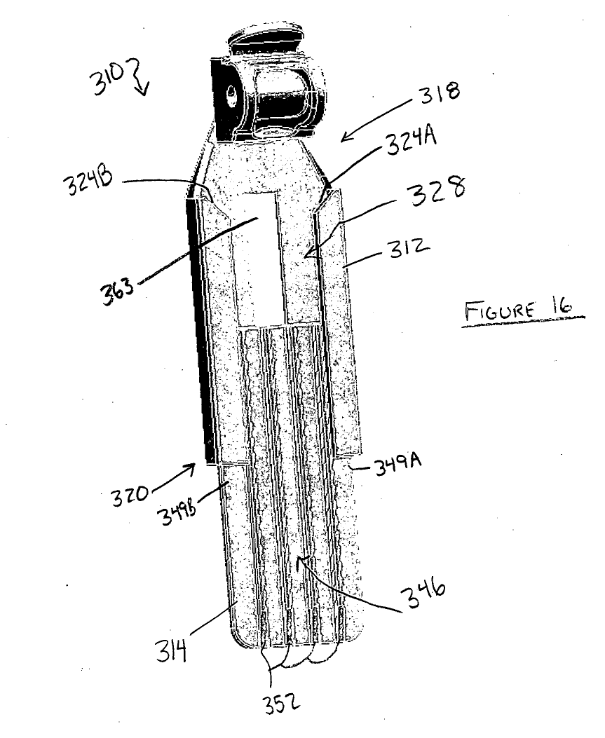

The adjustable blade 314 may include an adjustment mechanism that engages the fixed blade 312 to facilitate adjustment of the adjustable blade 314 relative the fixed blade 312. In the illustrated embodiment, for example, the adjustable blade 314 includes a pair of prongs 361 that extend from the inner surface 344 of the adjustable blade 314 to engage a longitudinally oriented slot 363 provided in the fixed blade 312. Each prong 361A, B extends through the slot 363 and includes a flange 365A, B that engages the outer surface of the fixed blade 312 to inhibit separation of the adjustable blade 314 from the fixed blade 312. The prongs 361A, B may be flexible and resilient. For example, the prongs 361A, B may be compressed toward one another from an expanded configuration, illustrated in FIG. 18, to a collapsed position. In the expanded configuration, the prongs 361A, B may engage the sidewalls of the slot 363 to inhibit adjustment of the adjustable blade 314 relative to the fixed blade 312. In a collapsed position, the prongs 361A, B may be disengaged from the sidewalls of the slot 363 to facilitate adjustment of the adjustable blade 314 relative to the fixed blade 312. The prongs 361A, B may be biased to the expanded configuration, as in the illustrated embodiment.

FIGS. 19-22B illustrate an exemplary embodiment of an instrument 500 for adjusting an adjustable blade of a retractor blade assembly. The exemplary instrument 500 will be described in connection with the retractor blade assembly 10 described above in connection with FIGS. 1-7, although one of skill in the art will appreciate that the exemplary instrument 500 may be used with any type of retractor blade assembly, including any of the exemplary embodiments of the retractor blade assemblies disclosed herein. In the illustrated embodiment, the instrument 500 includes a handle 502 positioned at a proximal end of the instrument 500 and an instrument body 504 that extend distally from the handle 502 to terminate at a distal end 506 of the instrument 500. The illustrated instrument 500 is bayoneted, e.g., the longitudinal axis of the handle 500 is offset from the longitudinal axis of a distal portion of the instrument body 504, to minimize obstruction of the working channel of the retractor blade assembly during use. The handle 502 may be offset by any amount, depending, for example, on the retractor blade assembly with which the instrument is designed for use, although, one of ordinary skill in the art will appreciate that the instrument 500 need not be bayoneted and that in certain embodiments the handle 502 and the instrument body 504 may be coaxial.

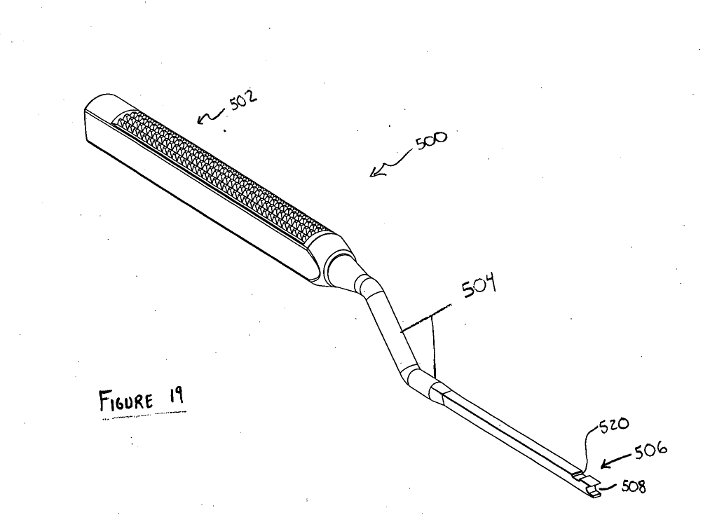

The distal end 506 of the instrument 500 may be configured to facilitate engagement of the instrument 500 with an adjustable blade of a retractor blade assembly. For example, in the illustrated embodiment, the distal end 500 has a distal facing notch 508 formed therein for engagement with, for example, a portion of the tab 60 of the adjustable blade 14. The distal notch 508, in the illustrated embodiment, is defined by a first surface 510 and a second surface 512. The first surface 510 extends proximally from the distal end 506 and is oriented generally parallel to the longitudinal axis of the distal end 506 of the instrument body 504 and a top surface 514 of the instrument body 504. The second surface 512 is angled proximally from the distal end 508 toward the first surface 510. The orientation of the second surface 512 and the first surface 510 may be varied depending on the geometry of the retractor blade assembly with which the instrument 500 is design for use. For example, in the illustrated embodiment, the second surface 512 is oriented at an angle to the bottom surface 516 of the instrument body 504 to facilitate engagement of the distal end 508 with the tab 60 of the adjustable blade 14 of the exemplary retractor blade assembly 10. In particular, the orientation of the second surface 512 is selected to move the tab 60 from the first position, in which the projection 72 engages one of the teeth 30 of the fixed blade 12, to the second position, in which the tab 60 is moved away from the fixed blade 12 and the projection 72 is disengaged from the tooth, as the distal end 506 of the instrument 500 is advanced distally. The angle of orientation of the second surface 512, indicated by angle A in FIG. 22B, may be any acute angle. In certain exemplary embodiments, the angle A may be approximately 20° to approximately 40°. In one exemplary embodiment, the angle A is approximately 25°.

In certain exemplary embodiments, including the illustrated embodiment, the distal notch 508 may include a third surface 518 that is interposed between the first surface 510 and the second surface 512. In the illustrated embodiment, the third surface 518 is oriented generally perpendicular to the longitudinal axis of the distal end 506 of the instrument body 504 to facilitate engagement of the third surface 518 with the proximally facing instrument engagement surface 66 of the tab 60. The orientation of the third surface 518 may be varied depending on, for example, the orientation of the surface with which the third surface 518 is design to engage. The third surface 518, in the illustrated embodiment, is arcuate in shape, although the third surface 518 may be linear, angled or other suitable shapes. In alternative exemplary embodiments, the first surface 510 and the second surface 512 may intersect, in which case a third surface will not be present.

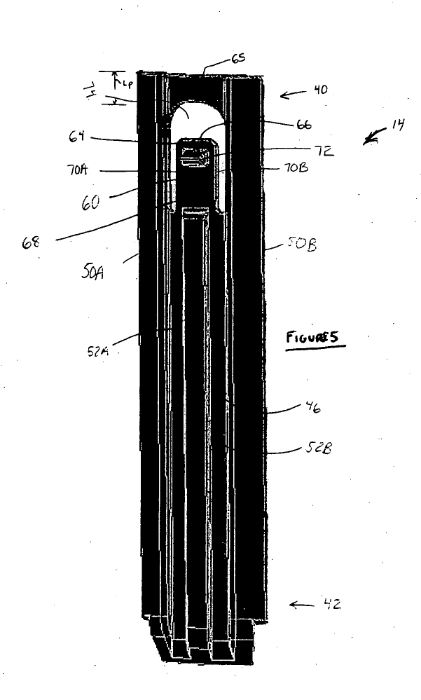

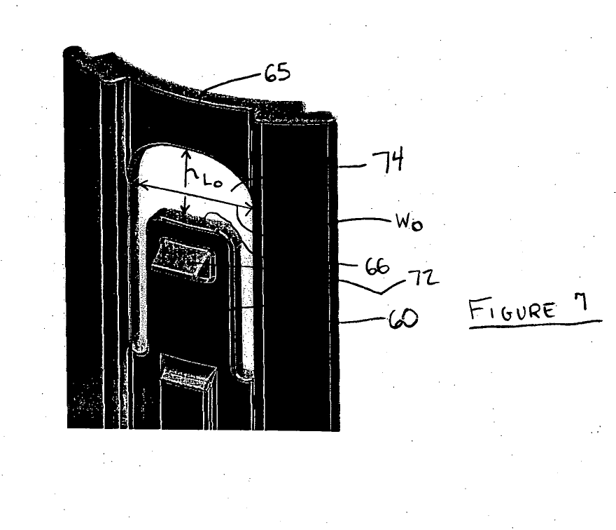

Continuing to refer to FIGS. 19-22B, in particular FIGS. 22A-22B, the instrument body 504 may include a cut-out formed therein to facilitate engagement of the distal end 506 with an adjustable blade of a retractor blade assembly. In the illustrated embodiment, for example, the distal end 506 of the instrument body 504 includes a cut-out 520 that is formed in the bottom surface 516 of the instrument body 504 and is positioned proximal to the notch 508. The cut-out 520 may be configured, e.g., sized, shaped, and positioned, to engage a portion of an adjustable blade to facilitate proximal adjustment of the adjustable blade relative to the fixed blade. In the illustrated embodiment, for example, the cut-out 520 is configured to engage a proximal edge 65 of the adjustable blade 14 (FIGS. 5 & 7). In particular, the length L_{C} of the cut-out 520 (FIG. 22B) may be greater than or equal to the length L_{P} of the proximal edge 65 of the adjustable blade 14 (FIG. 5) and the cut-out 520 may be proximally offset from the distal end 506 of the instrument body 504 a length L_{I}, which may be greater than or equal to the L_{O} of the opening 74 in the adjustable blade 14 (FIG. 7).

The instrument 500 may be constructed from any material suitable for use in vivo, including, for example, metal, such as stainless steel or titanium, polymers, ceramics, or combinations thereof.

In use, the distal end 506 of the instrument body 504 may be engaged to the adjustable blade 14 of the retractor blade assembly 10 to facilitate proximal and distal adjustment of the adjustable blade 14 relative to the fixed blade 12. In particular, distal end 504 is advanced distally to position the proximal end of the tab 60 within the notch 508. As the proximal end of the tab 60 is advanced into the notch 508, the second surface 512 causes the tab 60 to move from the first position to the second position to disengage the projection 72 from a tooth of the fixed blade 12, thereby freeing the adjustable blade 14 to move relative to the fixed blade 12. The distal end 506 is advanced distally until the proximal edge 65 is positioned within the cut-out 520 and the third surface 518 abuts the proximally facing surface 66 of the tab 60. Once the instrument 500 is positioned, the adjustable blade 14 may be adjusted, proximally or distally, using the instrument. The instrument 500 can be used to adjust the adjustable blade 14 prior to or during a surgical procedure.

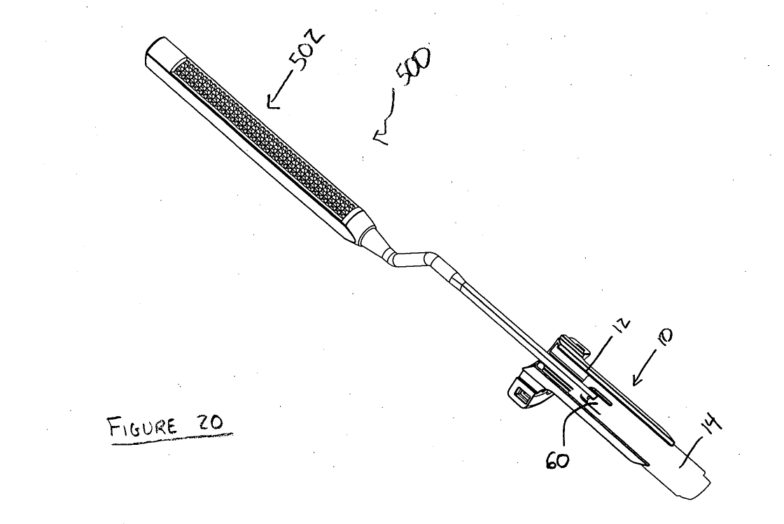

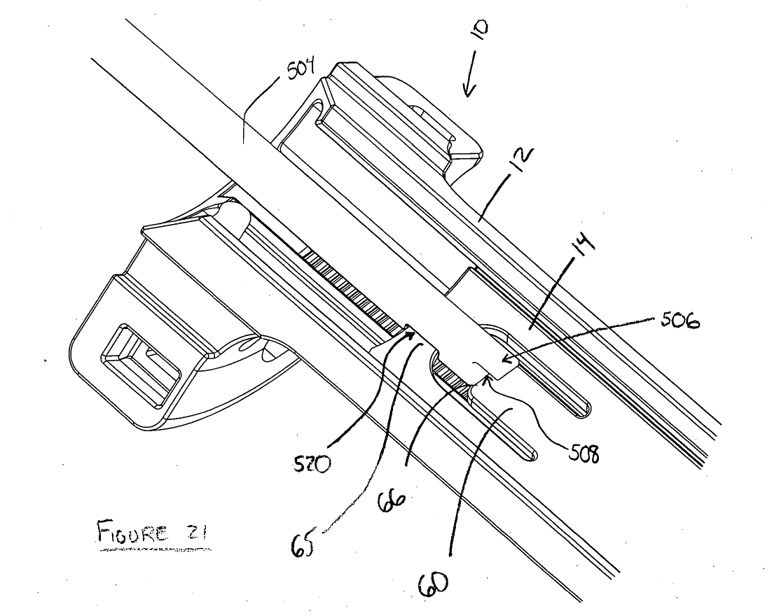

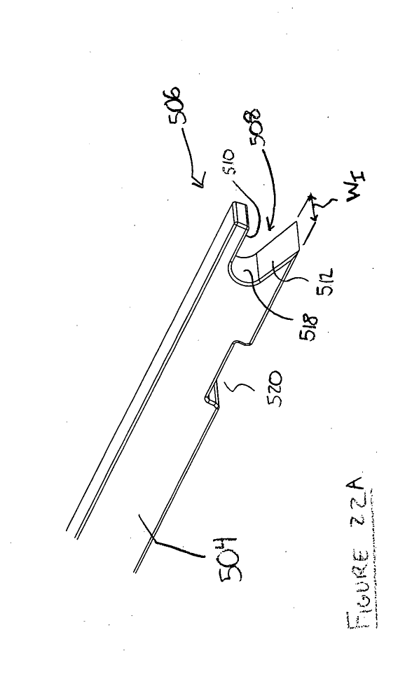

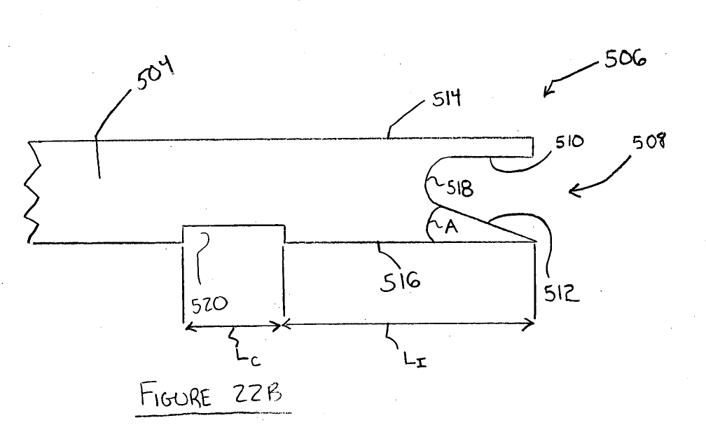

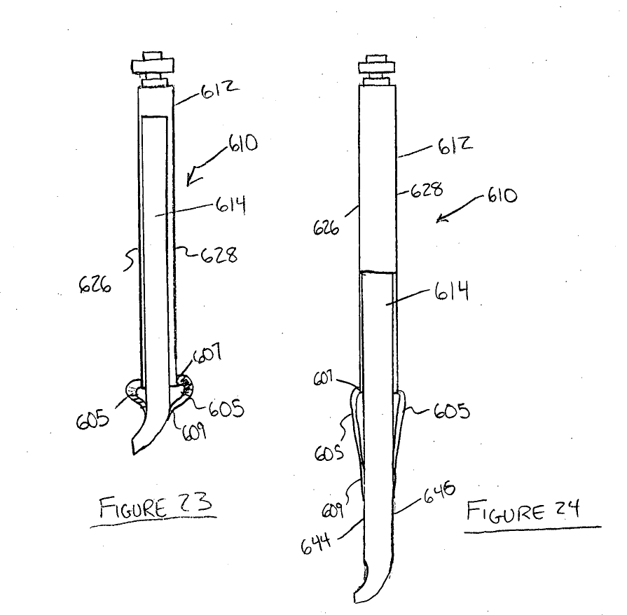

FIGS. 23 and 24 illustrate another embodiment of a retractor blade assembly 610 having a fixed blade 612 and an adjustable blade 614 that is longitudinally adjustable relative to the fixed blade 612. The exemplary retractor blade assembly 610 includes a flexible sheath 605 coupled at a first portion 607 to the fixed blade 612 and at a second portion 609 to the adjustable blade 614. In the exemplary embodiment, the flexible sheath 605 is coupled at one end to the fixed blade 612 and at another end to the adjustable blade 614, although any portion or portions of the flexible sheath 605 may be coupled to the blades. The flexible sheath 605 may be connected at any point along the length of the fixed blade 612 or the adjustable blade 614. In the illustrated embodiment, for example, the flexible sheath 605 is connected to the distal end of the fixed blade 612 and at the distal end of the adjustable blade 614.

The flexible sheath 605 may be configured to inhibit tissue impingement between the adjustable blade 614 and the fixed blade 612, particularly when the adjustable blade 614 is adjusted proximally and withdrawn into the fixed blade 612. The flexible sheath 605 is preferably connected to the outer surface 646 of the adjustable blade 614 and the outer surface 628 of the fixed blade 612. In the illustrated embodiment, the flexible sheath 605 is connected to both the inner surface (626, 644) and the outer surface (628, 646) of the fixed blade 612 and the adjustable blade 614, respectively. The flexible sheath 605 may span at least a substantial portion of the width of the outer surface 646 of the adjustable blade 614. In the illustrated embodiment, the flexible sheath 605 is tubular in shape and is positioned to surround the adjustable blade 614.

The flexible sheath 605 may be constructed from any flexible material, including, for example, flexible polymers. In certain exemplary embodiments, the flexible sheath 605 may be constructed from an elastomeric polymer. The flexible sheath 605 may be constructed to be expandable from a generally collapsed configuration, as illustrated in FIG. 23, to an expanded configuration, illustrated in FIG. 24, during adjustment of the adjustable blade 614 to a distal position. In the expanded configuration, the flexible sheath 605 may be stretched along the surfaces of the adjustable blade 614 to inhibit tissue from entering the fixed blade 612.

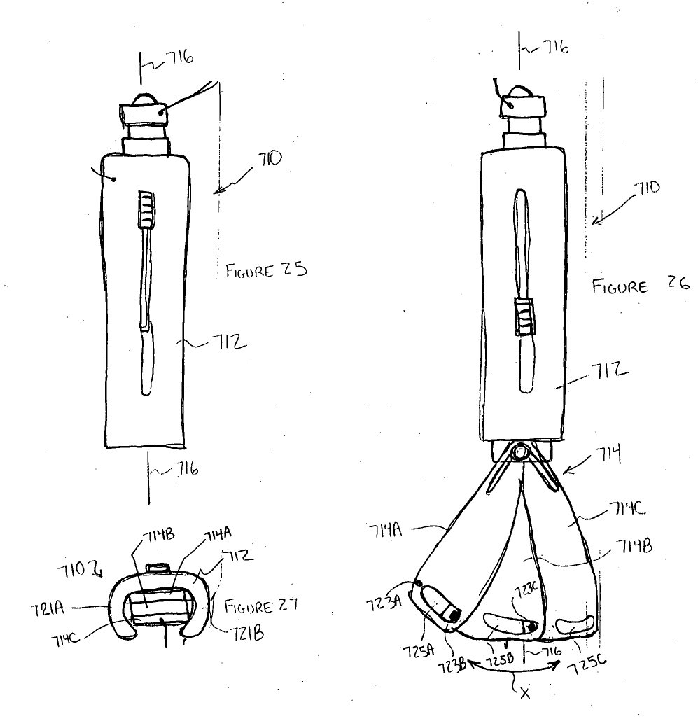

FIGS. 25-29 illustrate another exemplary embodiment of a retractor blade assembly 710 including a fixed blade 712 and an adjustable blade assembly 714 that is adjustable along the longitudinal axis 716 of the fixed blade 712. FIG. 25 illustrates the adjustable blade assembly 714 in a proximal, retracted position in which a substantial portion of the adjustable blade assembly 714 is positioned within the fixed blade 712. FIG. 26 illustrates the adjustable blade assembly 714 in a distal, expanded position in which a portion of the adjustable blade assembly 714 is withdrawn from the fixed blade 712 thereby increasing the overall length of the retractor blade assembly 710.

In the certain exemplary embodiments, including the illustrated embodiment, the adjustable blade assembly 714 comprises a plurality of laterally adjustable blades that are adjustable relative to one another in a direction transverse to the longitudinal axis 716 of the fixed blade 712 when the adjustable blade assembly 714 is advanced to a distal position, as illustrated in FIG. 25. In the illustrated embodiment, for example, the adjustable blade assembly 714 comprises three adjustable blades 714A, 714B, & 714C, each of which is adjustable longitudinally relative to the longitudinal axis 716 of the fixed blade 712 and at least two of which, adjustable blades 714A and 714C, are also adjustable relative to one another along an arcuate axis X that is oriented transverse to the longitudinal axis 716 of the fixed blade 712. The adjustable blade assembly 714 may include any number of laterally adjustable blades of any size or shape, depending on, for example, the desired width of the retractor blade assembly in the expanded configuration. Moreover, the amount of lateral expansion and the orientation of the laterally adjustable blades may be varied depending on, for example, the desired width of the retractor blade assembly in the expanded configuration.

Continuing to refer to FIGS. 25-29, the adjustable blades 714A-C may be adjustable from a laterally contracted configuration, illustrated in FIG. 25, to a laterally expanded configuration, illustrated in FIG. 26. In the illustrated embodiment, for example, the adjustable blades 714A-C assume a laterally contracted configuration when the adjustable blade assembly 714 is in a proximal position and expand laterally when the adjustable blade assembly 714 is advanced to a distal position. The adjustable blades 714A-C, in the illustrated embodiment, are interconnected at pivot point 715 such that the laterally adjustable blades 714A & 714C expand laterally in the manner of a fan. The laterally adjustable blades 714A & 714C are pivotable about the pivot point 715 from a laterally contracted or collapsed position, in which the longitudinal axis of each adjustable blades 714A-C are oriented approximately parallel to the longitudinal axis 716 of the fixed blade 712, to a laterally expanded position, in which the longitudinal axis of each of the laterally adjustable blades 714A & 714C is oriented at an angle to the longitudinal axis 716 of the fixed blade 712. In certain exemplary embodiments, including the illustrated embodiment, the laterally adjustable blades 714A & 714C may be biased to the laterally expanded configuration. In the illustrated embodiment, for example, the adjustable blade assembly 714 includes a torsion spring 719 to bias the laterally adjustable blades to the laterally expanded configuration.

In certain exemplary embodiments, the fixed blade 712 may be configured to constrain the laterally adjustable blades in a laterally contracted or collapsed configuration. For example, in the illustrated embodiment, the fixed blade 712 has a generally C-shaped cross-section defined by two spaced apart arcuate sidewalls 721A,B, as illustrated in FIG. 27. The laterally adjustable blades 714A & 714C may be engaged by the sidewalls 721A,B to constrain the adjustable blade assembly 714 in the laterally contracted configuration when the adjustable blades 714A-C are at least partially withdrawn into the fixed blade 712.

In certain exemplary embodiments, in particular embodiments in which the laterally adjustable blades are biased to a laterally expanded configuration, the adjustable blade assembly 714 may include a mechanism to limit the lateral expansion of the laterally adjustable blades 714A & 714C. For example, in the illustrated embodiment, each of the adjustable blades 714A-C includes a pin 723 that is configured to engage a slot 725 in an adjacent adjustable blade. The slots 725 may be shaped and oriented to control the lateral adjustment of the laterally adjustable blades 714A and 714C. For example, in the illustrated embodiment, each slot 725 is arcuate in shape to facilitate lateral adjustment of the laterally adjustable blades 714A & 714C along an arcuate path. One skilled in the art will appreciate that the shape and orientation of the slots 725, as well as the position of the pins 723, may be varied depending on the extent of lateral adjustment desired.

FIG. 30 illustrates another exemplary embodiment of an adjustable blade assembly 814 that includes a plurality of adjustable blades 814A-814C, at least two of which, laterally adjustable blades 814A and 814C, are laterally adjustable relative to one another. In the illustrated embodiment, each adjustable blade 814A-C includes a pair of spaced apart raised edges 829, 831. The raised edges 829 are configured to engage raised edges 831 to limit lateral expansion of the laterally adjustable blades 814A & 814C.

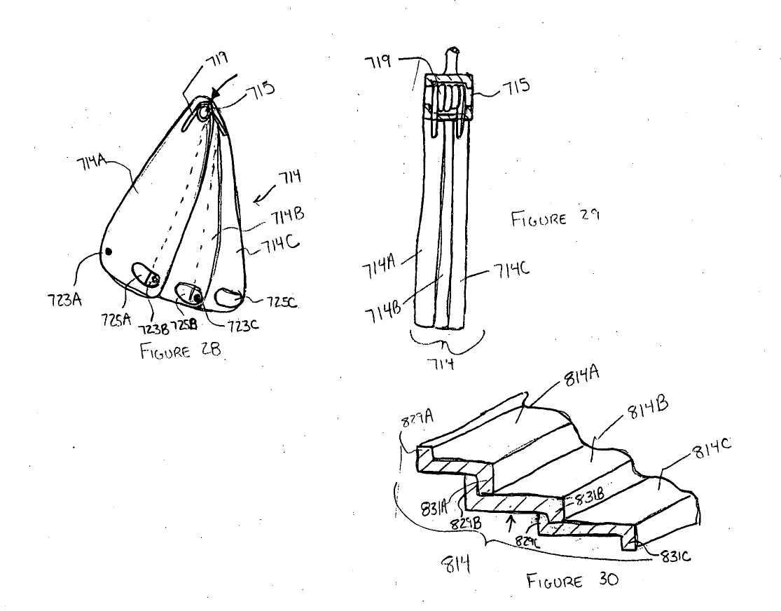

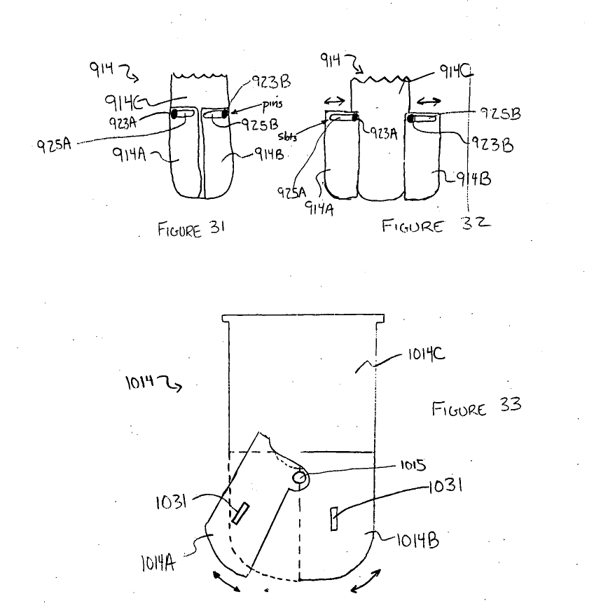

FIGS. 31 and 32 illustrate another exemplary embodiment of an adjustable blade assembly 914 that includes a plurality of laterally adjustable blades 914A-914B, each of which is laterally adjustable relative to another adjustable blade, for example, center adjustable blade 914C, which is not laterally adjustable, as well as each other. In the illustrated embodiment, the laterally adjustable blades 914A and 914B are independently laterally adjustable along an axis that is transverse to the longitudinal axis of the fixed blade. The center adjustable blade 914C includes two spaced apart pins 923A and 923B each of which engages a slot 925A, 925B in a respective one of the laterally adjustable blades 914A and 914B. The slots 925A, 925B are linear in shape and oriented generally perpendicular to the longitudinal axis of the fixed blade and the center adjustable blade 914C. One skilled in the art will appreciate that the slots 925A,B may have a variety of shapes and may be oriented in a variety of orientations, depending, for example, on the desired lateral width of the retractor blade assembly:

FIG. 33 illustrates another exemplary embodiment of an adjustable blade assembly 1014 that includes a plurality of laterally adjustable blades 1014A-1014B, which are laterally adjustable relative to one another. In the illustrated embodiment, the laterally adjustable blades 1014A and 1014B are independently laterally adjustable along an axis that is transverse to the longitudinal axis of the fixed blade. The laterally adjustable blades 1014A and 1014B, in the illustrated embodiment, are pivotally connected about a pivot pin 1015 that couples the laterally adjustable blades 1014A and 1014B to a central adjustable blade 1014C. One or more of the laterally adjustable blades 1014A and 1014B may include a slot 1031 or other structure to facilitate lateral adjustment of the laterally adjustable blades 1014A and 1014B, for example, with an instrument.

While the retractor blade assemblies, instruments, and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A retractor blade assembly comprising:
a fixed blade having a longitudinal axis; and
an adjustable blade operatively coupled to the fixed blade and adjustable relative to the fixed blade along the longitudinal axis of the fixed blade, the adjustable blade including a flexible tab, the flexible tab being movable between a first position in which the tab is generally co-planar to the adjustable blade and a second position in which the tab is oriented generally transverse to the adjustable blade.

2. The retractor blade assembly of claim 1, wherein the flexible tab is biased to the first position.

3. The retractor blade assemble of claim 2, wherein the flexible tab is generally rectilinear is shape.

4. The retractor blade assembly of claim 3, wherein a distal portion of the flexible tab is coupled to and formed from the adjustable blade.

5. The retractor blade assembly of claim 4, wherein the flexible tab is pivotable about the distal portion between the first and second positions.

6. The retractor blade assembly of claim 1, wherein the flexible tab is configured to move away from the fixed blade when moved to the second position.

7. The retractor blade assembly of claim 1, wherein the flexible tab includes a projection for engaging the fixed blade when the tab is in the first position.

8. The retractor blade assembly of claim 7, wherein the fixed blade includes a plurality of stops for receiving the projection.

9. The retractor blade assembly of claim 8, wherein the stops comprise a plurality of detents aligned longitudinally along the fixed blade.

10. The retractor blade assembly of claim 8, wherein the stops comprise a plurality of teeth aligned longitudinally along the fixed blade.

11. A retractor blade assembly comprising:
a fixed blade having a longitudinal axis; and
an adjustable blade operatively coupled to the fixed blade and adjustable relative to the fixed blade along the longitudinal axis of the fixed blade, the adjustable blade including a flexible tab, the flexible tab being pivotable between a first position, in which the tab is generally co-planar to the adjustable blade, to a second position, in which the tab is oriented generally transverse to the adjustable blade, the flexible tab having a projection engageable with one or more longitudinally aligned stops provided on the fixed blade, the projection engaging a stop when the tab is the first position to fix the adjustable blade relative to the fixed blade, the tab including a proximally facing instrument engagement surface to facilitate adjustment of the adjustable blade relative to the fixed blade.

12. The retractor blade assembly of claim 11, wherein the adjustable blade includes an opening adjacent the instrument engagement surface to facilitate positioning of an instrument against the instrument engagement surface.

13. The retractor blade assembly of claim 12, wherein the opening is sized to receive the distal end of an instrument for adjustment of the adjustable blade relative to the fixed blade.

14. The retractor blade assembly of claim 12, wherein the opening is positioned proximal to the instrument engagement surface.

15. The retractor blade assembly of claim 11, wherein the instrument engagement surface is oriented generally perpendicular to the longitudinal axis of the fixed blade.

16. A retractor blade assembly comprising:
a fixed blade having a longitudinal axis; and
an adjustable blade operatively coupled to the fixed blade and adjustable relative to the fixed blade along the longitudinal axis of the fixed blade, the adjustable blade including a flexible tab formed from the adjustable blade and having a distal end coupled to the adjustable blade, a proximal end of the flexible tab being pivotable about the distal end between a first position, in which the tab is generally co-planar to the adjustable blade, to a second position, in which the tab is oriented generally transverse to the adjustable blade, the flexible tab being biased to the first position and having a projection engageable with one or more longitudinally aligned stops provided on the fixed blade, the projection engaging a stop when the tab is the first position to fix the adjustable blade relative to the fixed blade, the proximal end of the tab including a proximally facing instrument engagement surface and the adjustable blade including an opening proximally adjacent the instrument engagement surface to facilitate positioning of an instrument against the instrument engagement surface.

17. An instrument for adjusting an adjustable blade of a retractor blade assembly, the instrument comprising:
a handle, and
an instrument body coupled to the handle, the instrument body including a distal end having a distal facing notch formed therein, the notch being defined by a first surface and a second surface, the second surface being angled proximally toward the first surface, wherein the notch is sized to receive a portion of the adjustable blade between the first surface and the second surface.

18. The instrument of claim 17, wherein the notch has a third surface interposed between and connecting the first and second surfaces.

19. The instrument of claim 18, wherein the third surface is arcuate in shape.

20. The instrument of claim 18, wherein the third surface is oriented generally perpendicular to a longitudinal axis of the instrument body.

21. The instrument of claim 17, wherein the instrument body includes a cut-out formed therein, the cut-out being positioned proximal to the notch and being sized to receive another portion of the adjustable blade therein.

22. An instrument for adjusting an adjustable blade of a retractor blade assembly, the instrument comprising:
a handle, and
an instrument body coupled to the handle, the instrument body having a top surface, a bottom surface and a distal end, the distal end having a distal facing notch formed therein, the notch being defined by a first surface and a second surface, the second surface being angled proximally from the bottom surface of the instrument body toward the first surface, the notch being sized to receive a portion of the adjustable blade between the first surface and the second surface, the bottom surface of the instrument body including a cut-out formed therein, the cut-out being positioned proximal to the notch and being sized to receive another portion of the adjustable blade therein.

23. A retractor blade assembly comprising:
a fixed blade having a longitudinal axis;
an adjustable blade operatively coupled to the fixed blade and adjustable relative to the fixed blade along the longitudinal axis of the fixed blade; and
a flexible sheath coupled at one portion to the fixed blade and at a second portion to the adjustable blade.

24. The retractor blade assembly of claim 23, wherein adjustable blade has an inner surface and an outer surface and the flexible sheath is coupled to the outer surface.

25. The retractor blade assembly of claim 24, wherein the flexible sheath is coupled to the inner surface and the outer surface.

26. The retractor blade assembly of claim 23, wherein the flexible sheath is expandable from a generally collapsed configuration to an expanded configuration upon adjustment of the adjustable blade in a distal direction relative to the fixed blade.

27. The retractor blade assembly of claim 26, wherein the sheath, in the expanded configuration, is generally tubular in shape.

28. A retractor blade assembly comprising:
a fixed blade having a longitudinal axis; and
a plurality of adjustable blades operatively coupled to the fixed blade and adjustable relative to the fixed blade along the longitudinal axis of the fixed blade between a proximal position and a distal position, the plurality of adjustable blades including one or more laterally adjustable blades that are adjustable relative to one another in a direction transverse to the longitudinal axis of the fixed blade when the plurality of adjustable blades are adjusted to a distal position.

29. The retractor blade assembly of claim 28, wherein the laterally adjustable blades are interconnected at a pivot point.

30. The retractor blade assembly of claim 29, wherein the laterally adjustable blades are pivotable about the pivot point from a collapsed position, in which the longitudinal axis of each laterally adjustable blade is oriented approximately parallel to the longitudinal axis of the fixed blade, to an expanded position, in which the longitudinal axis of each laterally adjustable blade is oriented at an angle to the longitudinal axis of the fixed blade.

31. The retractor blade assembly of claim 30, wherein the laterally adjustable blades are biased to the expanded position.

32. The retractor blade assembly of claim 31, further comprising a torsion spring coupled to the laterally adjustable blade to bias the laterally adjustable blades to the expanded position.

33. The retractor blade assembly of claim 28, wherein the fixed blade includes a pair of spaced apart channels, each of the channels receiving a rail provided on the adjustable blade.
